(19) United States
(12) Patent Application Publication
(10) Pub. No.: US 2009/0197774 A1
(43) Pub. Date: Aug. 6, 2009

(54) METHOD FOR DIAGNOSING THROMBOEMBOLIC DISORDERS AND CORONARY HEART DISEASE

(75) Inventors: Detlef Kozian, Frankfurt am Main (DE); Matthias Herrmann, Frankfurt am Main (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/089,624

(22) PCT Filed: Sep. 30, 2006
(86) PCT No.: PCT/EP0606/009517

§ 371 Date: Sep. 5, 2008

(30) Foreign Application Priority Data
Oct. 12, 2005 (DE) 10 2005 048 899.4

(51) Int. Cl. C12Q 1/68; G01N 33/53; C40B 30/04; C12Q 1/02

(52) U.S. Cl. 506/9; 435/6; 436/501; 435/7.92; 435/29

(57) ABSTRACT

The present invention refers to a method for the in vitro diagnosis of thromboembolic and/or coronary heart diseases, wherein the nucleotide at position 470 of a nucleic acid coding for the human EGLN2 protein or the amino acid at position 58 of the human EGLN2 protein of a sample of a person is determined.

The present invention refers to a method for the in vitro diagnosis of thromboembolic and/or coronary heart diseases, wherein the nucleotide at position 470 of a nucleic acid coding for the human EGLN2 protein or the amino acid at position 58 of the human EGLN2 protein of a sample of a person is determined.

EGLN2, due to its HIF prolyl hydroxylase activity also known as prolyl hydroxylase domain-containing protein 1 (PHD1), belongs to a group of closely related proteins of the Egl-Nine gene family which has a conserved genomic structure consisting of five coding exons. HIF (hypoxia-inducible factor) is a transcriptional regulator that plays a key role in many aspects of oxygen homeostasis but the contribution of the EGLN isoforms EGLN1 (PHD1), EGLN2 (PHD2) and EGLN3 (PHD3) to the physiological regulation of HIF is still uncertain (Appelhoff, R. J. et al. (2004) J. Biol. Chem., 279, 38458-38465, No. 37). It is reported that all EGLN isoforms show a differing cell specific and inducible behaviour, which should allow flexibility in the regulation of the HIF response to hypoxia. This would mean that specific pharmacological inhibition of a particular EGLN isoenzyme could have the potential for selective modulation of the HIF response that would be useful in therapeutic applications (Appelhoff, R. J. et al. (2004), supra). EGLN2 inhibition, for example, should activate the HIF response broadly across a range of cell types under resting conditions. In contrast specific inhibition of EGLN3 should selectively augment the response to hypoxia in certain tissues that express high levels of the enzyme (Appelhoff, R. J. et al. (2004), supra). This could open the possibility to treat ischemic/hypoxic diseases. Contrary to EGLN2 and EGLN3 not much is known for the physiological role of EGLN1.

In order to better understand a potential involvement of EGLN2 in the occurrence and progression of coronary heart diseases, genotype-phenotype association analyses have been carried out with a well characterized patient group with respect to a variation in the EGLN2 gene in position 470 of the EGLN2 reference sequence published under the reference number NM_{—}053046.2 in accordance with the present invention. Different genetic variants of the EGLN2 gene are already known as SNPs (single nucleotide polymorphisms) and published under

http://www.ncbi.nlm.nih.gov/SNP/snp_ref.cgi?locusId=112398).

Surprisingly it has been found that a variation of the nucleotide at position 470, in particular from cytosine to thymidine of a nucleic acid coding for the human EGLN2 protein or the amino acid at position 58, in particular from serine to leucine of the human EGLN2 protein correlates with the occurrence of thromboembolic and/or coronary heart diseases.

Therefore, a subject matter of the present invention relates to an in vitro or in vivo diagnosis of thromboembolic and/or coronary heart diseases, wherein the nucleotide at position 470 of a nucleic acid coding for the human EGLN2 protein or the amino acid at position 58 of the human EGLN2 protein of a sample of a person or patient is determined.

In a preferred embodiment of the present invention the thromboembolic disease is stroke, a prolonged reversible ischemic neurological deficit (PRIND) and/or a transitoric ischemic attack (TIA) and the coronary heart disease is a myocardial infarction.

In particular, if the nucleotide at position 470 is determined as thymidine in the chromosomal DNA or uracile in the mRNA or the amino acid at position 58 is determined as leucine there exists a higher risk of stroke, PRIND and/or TIA. If, however, the nucleotide at position 470 is determined as cytidine or the amino acid at position 58 is determined as serine there exists a higher risk for a myocardial infarction, in particular early myocardial infarction.

According to the present invention, the term “EGLN2-C470C” refers to the group of persons which have cytidine on both alleles of the gene coding for EGLN2 at position 470 of the reference sequence NM_{—}053046.2 which leads to the amino acid serine at position 58 of the corresponding protein. These persons are homozygous with respect to this EGLN2 variant. Consequently, the term “EGLN2-C470T refers to the group of persons which have cytidine on one allele of the gene coding for EGLN2 which leads to serine at position 58 of the corresponding protein and thymidine on the other allele of the gene coding for EGLN2 which leads to leucine at position 58 of the corresponding protein. These persons are heterozygous with respect to this EGLN2 variant.

The nucleic acid sequence of the reference sequence coding for the human EGLN2 protein preferably has the nucleic acid sequence of SEQ ID NO: 1 and the amino acid sequence of the human EGLN2 protein preferably has the amino acid sequence of SEQ ID NO: 2. However, the present invention encompasses also other variants of human EGLN2 and the non-human homologs thereof, as for example other mammalian EGLN2 homologs or the EGLN2 homologs from Caenorhabdidis elegans, mouse or rat, provided that there is a nucleotide exchange from cytidine to thymidine at the position corresponding to position 470 of said reference sequence and/or an amino acid exchange from serine to leucine at the position corresponding to position 58 of said reference sequence and further provided that the corresponding protein has a prolyl hydroxylase activity, in particular a HIF prolyl hydroxylase activity. Said enzyme activity can be measured for example by mass spectrometric analysis whereby the oxidization of Pro, e.g. Pro⁵⁶⁴ of HIF-1α, can be detected, or by enzymatic assays known to a person skilled in the art.

Generally, the specific nucleotide at position 470 can be determined by a nucleic acid sequencing method, a mass spectrometric analysis of the nucleic acid, a hybridisation method and/or an amplification method. Examples of a nucleic acid sequencing method are pyrosequencing and/or sequencing with the help of radioactive and/or fluorescence labelled nucleotides. Examples of the hybridisation method are Southern blot analysis, Northern blot analysis and/or a hybridisation method on a DNA-microarray. Examples of an amplification method are a TaqMan analysis, a differential RNA display analysis and/or a representational difference analysis (Shi M. M. (2002) Am J Pharmacogenomics., 2 (3), 197-205; Kozian & Kirschbaum (1999) Trends Biotechnol., 17 (2), 73-8.)

Furthermore, the amino acid sequence at position 58 can be determined by a method measuring the amount of the specific protein and/or a method measuring the activity of the specific protein. Examples of a method for measuring the amount of the specific protein are a Western blot analysis and/or an ELISA. Examples for measuring the activity of the specific protein are an in vitro test assay and/or an in vitro whole cell test assay with human cells, animal cells, bacterial cells or yeast cells, all known to a person skilled in the art.

Examples of a sample for the detection of the respective variant are a cell, a tissue or a body fluid, in particular in cellular components of the blood, endothelial cells or smooth muscle cells. Preferably the sample is pre-treated by conventional methods known to a person skilled in the art in order to isolate and/or purify the nucleic acids or chromosomal DNA, or the proteins of the sample for the further analysis.

In an optional further step the risk and/or the age of a person to suffer from a thromboembolic and/or coronary heart disease can be determined as shown in the examples.

In another optional further step the dosage of a pharmaceutical against a thromboembolic and/or coronary heart disease can be determined.

In general, the found genetic variation in the EGLN2 gene can be used in accordance with the present invention as a genetic marker for the risk assessment and/or the prophylactic treatment of a thromboembolic and/or coronary heart disease (also known as “cardiovascular disease”), in particular of an early myocardial infarction, stroke, PRIND, TIA and/or coronary heart diseases.

Furthermore, the genetic variation can be used in accordance with the present invention as a genetic marker for the adaptation of the dosage of an effective therapeutic agent for the treatment of a person, individual or patient and/or for the identification of persons, individuals or patients being under or selected to be under clinical trial studies with an increased risk for a thromboembolic and/or coronary heart disease, in particular of an early myocardial infarction, stroke, PRIND, TIA and/or coronary heart diseases. The genetic variation can also be used in accordance with the present invention for the evaluation of the tolerance, safety and efficacy of a pharmaceutically active substance for a specific person, individual or patient or for identifying the person, individual or patient suitable for a particular treatment of said diseases.

In addition, the genetic variation can also be used in accordance with the present invention as part of a high throughput-screening assay for the detection and evaluation of pharmaceutically active compounds for the treatment of said diseases.

The present invention can also be used to identify risk factors for said diseases for each person, individual or patient to be treated or advised.

A preferred method for the diagnosis of a thromboembolic and/or coronary heart disease in accordance with the present invention contains the following steps:
(a) obtaining a sample, in particular a cell, tissue, body fluid, a cellular component of the blood, endothelial cells or smooth muscle cells, from a person or patient that should be investigated;
(b) isolating a nucleic acid probe, in particular a DNA probe from said sample;
(c) amplifying the specific region encompassing position 470 of the ENGL2 gene with the help of primers, in particular the primers as specified in the Examples;
(d) sequencing the amplified region;
(e) analysing the sequenced region; and
(d) assessing the risk for a thromboembolic and/or coronary heart disease, in particular for an early myocardial infarction, stoke, PRIND, TIA and/or coronary heart diseases.

An alternative method for the diagnosis of a thromboembolic and/or coronary heart disease in accordance with the present invention contains the following steps:
(a) obtaining a sample, in particular a cell, tissue, body fluid, a cellular component of the blood, endothelial cells or smooth muscle cells, from a person or patient that should be investigated;
(b) isolating the ENGL2 protein from said sample;
(c) determining the amino acid at position 58 of the EGLN2 protein; and
(d) assessing the risk for a thromboembolic and/or coronary heart disease, in particular for an early myocardial infarction, stoke, PRIND, TIA and/or coronary heart diseases.

More preferred steps are individually or collectively specified in the Examples and are incorporated hereby by reference to each step.

The following Figures, Tables, Sequences and Examples shall explain the present invention without limiting the scope of the invention.

### DESCRIPTION OF THE FIGURES

FIG. 1 shows the nucleic acid sequence of the human EGLN2 gene with the NCBI number NM_{—}053046. The primers used for amplification of the genetic section with the genetic variation C→T at position 470 (bold face) are underlined.

FIG. 2 shows the amino acid sequence of the human EGLN2 derived from the nucleic acid sequence with the NCBI number NM_{—}053046. The amino acid position 58 in the EGLN2 protein is in bold face.

FIG. 3 shows the influence of the genotype of EGLN2 at position 470 of the reference sequence NM_{—}053042.2, leading to amino acid exchanges at position 58 of the EGLN2 protein, on the age of the occurrence of coronary heart diseases in the patients group. P-values less than 0.05 are statistically relevant.

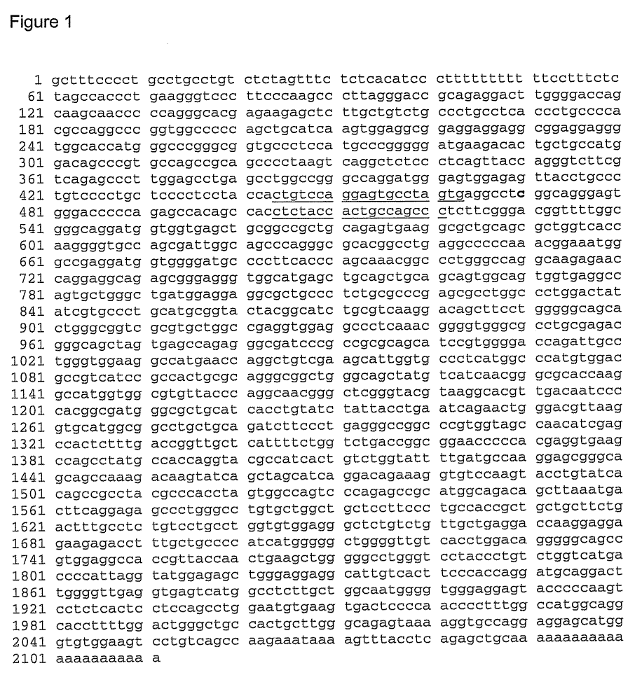

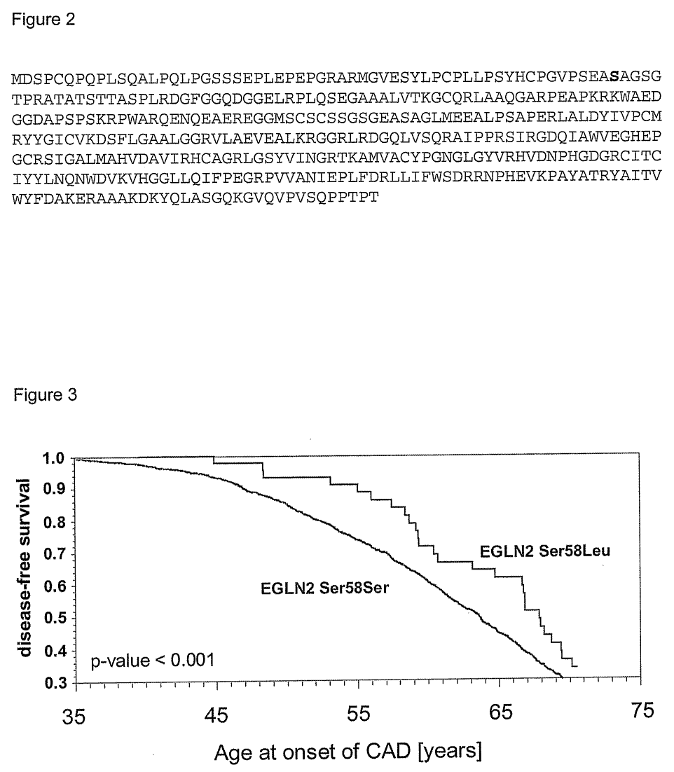

### DESCRIPTION OF THE SEQUENCES

SEQ ID NO: 1. shows the nucleic acid sequence of the human EGLN2 protein with the NCBI number NM_{—}053046.

SEQ ID NO: 2 shows the amino acid sequence of the human EGLN2 derived from the nucleic acid sequence with the NCBI number NM_{—}053046.

SEQ ID NO 3: shows the first primer sequence of nucleotides 444-463 of the reference sequence NM_{—}053046.2.

SEQ ID NO 4: shows the second primer sequence of complementary sequence of bases 504-521 of the reference sequence NM_{—}053046.2.

### EXAMPLES

### SNP Detection by Sequence and Analysis

### Oligonucleotides (Primers) for Amplification:

The following primers were used for the detection of the nucleotide exchange from C to T at position 470 in the EGLN2 sequence with the reference number NM_{—}053046.2:

Primer 1: 5′-CTGTCCAGGAGTGCCTAGTG-3′ (nucleotides 444-463 of the reference sequence NM_{—}053046.2; SEQ ID NO: 3);

Primer 2: 5′-GGGCTGGCAGTGGTAGAG-3′ (complementary sequence of bases 504-521 of the reference sequence NM_{—}053046.2; SEQ ID NO: 4).

### PCR Protocol for Amplification:

The reagents used were from Applied Biosystems (Foster City, USA): 20 ng of genomic DNA; 1 unit TaqGold DNA polymerase; 1×Taq polymerase buffer; 500 μM dNTPs; 2.5 mM MgCl₂; 200 nM of each amplification primer pair as shown above; H₂O ad 5 μl.

### Amplification Program of the PCR for Genotyping:

95° C. for 10 min×1 cycle
95° C. for 30 sec
70° C. for 30 sec×2 cycles;
95° C. for 30 sec
65° C. for 30 sec×2 cycles;
95° C. for 30 sec
60° C. for 30 sec×2 cycles;
95° C. for 30 sec
56° C. for 30 sec
72° C. for 30 sec×40 cycles;
72° C. for 10 min
4° C. for 30 sec×1 cycle;

### Protocol for Minisequencing and Detection of SNPs

The reagents used were from Applied Biosystems (Foster City, USA). 2 μl purified PCR product, 1.5 μl BigDye-Terminator-Kit, 200 nM of a sequencing primer as shown above; H₂O ad 10 μl.

### Amplification Program for Sequencing:

96° C. for 2 min×1 cycle;
96° C. for 10 sec
55° C. for 10 sec
65° C. for 4 min×30 cycles;
72° C. for 7 min
4° C. for 30 sec×1 cycle;

### Analyse of the Sequencing Products:

The sequences were analysed with the Sequenz Analyse Software (Applied Biosystems, Foster City, USA) for obtaining preliminary data first, then processed with the software Phred, Phrap, Polyphred und Consed. Phred, Phrap, Polyphred und Consed, written by Phil Green of the Washington University (http://www.genome.washington.edu).

### Results

### Characteristics of the Group of Persons

Table 1 shows the characteristics of the group of persons studied.

TABLE 1

| | | n | % |
|---|---|---|---|
| Total | | 2074 | |
| Sex | Female | 603 | 29.07 |
| | Male | 1471 | 70.93 |
| Age | | 61.8 (+/−10.5) | |
| BMI (Body Mass Index) | | 29.1 (+/−4.4) | |
| Blood Preasure | | 1214 | 58.7 |
| Smoker | | 1372 | 66.41 |
| Typ II Diabetis | | 361 | 17.46 |
| Myocardial infarction | | 830 | 40.59 |
| Stroke | | 145 | 7.01 |

### Frequence and Distribution of the Variants of the EGLN2 Gene

Table 2 shows the frequency and distribution of the genetic variants of the EGLN2 gene at position 470 of the reference sequence NM_{—}053046.2 in the patient group studied.

TABLE 2

| | Frequeny | Percentage |
|---|---|---|
| EGNL2-C470C | 1253 | 96.31 |
| (EGNL2 Ser58Ser) | | |
| EGNL2-C470T | 47 | 3.61 |
| (EGNL2 Ser58Leu) | | |
| EGNL2-T470T | 1 | 0.08 |
| (EGNL2 Leu58Leu) | | |
| Missing values | 773 | |

In the following only individuals with EGLN2-C470C (EGLN2 Ser58Ser) and EGLN2-C470T (EGLN2 Ser58Leu) are taken into account.

### Influence of the Variants of EGLN2 on the Occurrence of Early Myocardial Infarction

Table 3 shows the influence of the genotype of EGLN2 at position 470 of the reference sequence NM_{—}053046.2 on the occurrence of early myocardial infarction (less than 55 years old for men and less than 60 years old for women) and of stroke/PRIND (prolonged reversible ischemic neurological deficit)/TIA (transitoric ischemic aftack) in the patient group studied. P-values less than 0.05 are statistically relevant.

TABLE 3

EGLN2

| | C470C/ | C470T/ | |
|---|---|---|---|
| | Ser58Ser | Ser58Leu | |
| Clinical Parameter | n (%) | n (%) | p-value |
| Patients with early | 215 | 2 | 0.0199 |
| myocardial infarction | (17.16) | (4.26%) | |
| (<55m/60f) | | | |
| Patients without early | 1038 | 45 | |
| myocardial infarction | (82.84) | (95.74) | |
| (<55m/60f) | | | |
| Patients with | 88 | 7 | 0.0418 |
| Stroke/PRIND/TIA | (7.23) | (14.89) | |
| Patients without | 1165 | 40 | |
| Stroke/PRIND/TIA | (92.77) | (85.11) | |

### Results:

1. The patients with EGLN2-C470C showed a statistically higher incidence for early myocardial infarction compared to patients with EGLN2-C470T.
2. The patients with EGLN2-C470T showed a significant increase of the risk to receive a stroke, PRIND and/or TIA compared with patients with EGLN2-C470C.
   Influence of the Variants of EGLN2 on Patients' Age with Coronarv Heart Diseases

FIG. 3 shows the influence of the genotype of EGLN2 at position 470 of the reference sequence NM_{—}053042.2 on the age of the occurrence of coronary heart diseases in the patients group.

### Result:

A significant dependency of the age of the patients with EGLN2-C470C (EGLN2 Ser58Ser) for the early occurrence of coronary heart diseases was discovered compared to the age of patients with EGLN2-C470T (EGLN2 Ser58Leu).

### CONCLUSION

The statistically significant associations between the genetic variants of the gene coding for EGLN2 and/or the protein EGLN2 shown above are a clear indication for the involvement of said genetic variants in the occurrence of thrombotic and/or coronary heart diseases. Consequently, said genetic variants are biological markers for the prognosis of thrombotic and/or coronary heart diseases, in particular for the prognosis of early myocardial infarction and/or stroke, PRIND and/or TIA.

1. A method for the in vitro diagnosis of thromboembolic and/or coronary heart diseases, wherein the nucleotide at position 470 of a nucleic acid coding for the human EGLN2 protein or the amino acid at position 58 of the human EGLN2 protein of a sample of a person is determined.

2. The method of claim 1, wherein the thromboembolic disease is selected from the group consisting of stroke, prolonged reversible ischemic neurological deficit (PRIND) and/or transitoric ischemic attack (TIA).

3. The method of claim 1, wherein the coronary heart disease is myocardial infarction.

4. The method of claim 2, wherein the nucleotide at position 470 is determined as thymidine in the chromosomal DNA or uracile in the mRNA or the amino acid at position 58 is determined as leucine for a risk of stroke, PRIND and/or TIA.

5. The method of claim 3, wherein the nucleotide at position 470 is determined as a cytidine or the amino acid at position 58 is determined as serine for a risk of myocardial infarction.

6. The method according to claim 1 wherein the nucleic acid coding for the human EGLN2 protein has the nucleotide sequence of SEQ ID NO: 1.

7. The method according to claim 1 wherein the human EGLN2 protein has the amino acid sequence of SEQ ID NO: 2.

8. The method according to claim 1 wherein the nucleotide at position 470 is determined by a method selected from the group consisting of a nucleic acid sequencing method, a mass spectrometric analysis of the nucleic acid, a hybridisation method and an amplification method.

9. The method of claim 8, wherein the nucleic acid sequencing method is selected from the group consisting of pyrosequencing, sequencing with the help of radioactive and fluorescence labelled nucleotides.

10. The method of claim 8, wherein the hybridisation method is selected from the group consisting of Southern blot analysis, Northern blot analysis and a hybridisation method on a DNA-microarray.

11. The method of claim 8, wherein said amplification method is selected from the group consisting of a TaqMan analysis, a differential RNA display analysis and a representational difference analysis.

12. The method according to claim 1 wherein the amino acid sequence at position 58 is determined by a method selected from the group consisting of a method measuring the amount of the specific protein and a method measuring the activity of the specific protein.

13. The method according to claim 12, wherein the amount of the specific protein is measured by a method selected from the group consisting of a western blot analysis and an ELISA.

14. The method according to claim 12, wherein the activity of the specific protein is measured by in vitro test assay and an in vitro whole cell test assay using human cells, animal cells, bacterial cells or yeast cells.

15. The method according to claim 1, wherein said sample is selected from the group consisting of a cell, a tissue and a body fluid.

16. The method according to claim 1 wherein in a further step the risk of a person to suffer from a thromboembolic and/or coronary heart disease is determined.

17. The method according to claim 1 wherein in a further step the dosage of a pharmaceutical is determined.

18. A method for the in vitro diagnosis of thromboembolic and/or coronary heart diseases comprising the steps of
(a) obtaining a sample from a person wherein said sample is selected from the group consisting of a cell, a tissue and a body fluid:
(b) isolating a nucleic acid probe, in particular a DNA probe from said sample;
(c) amplifying a specific region encompassing position 470 of the ENGL2 gene with the help of primers;
(d) sequencing the amplified region;
(e) analysing the sequenced region; and
(d) assessing the risk for a thromboembolic and/or coronary heart disease, in particular for a myocardial infarction, stroke, PRIND, TIA and/or coronary heart diseases wherein the nucleotide exchange from cytidine to thymidine at the position corresponding to position 470 is indicative of an increased risk of said disease.

19. The method according to claims 5 or 18, wherein the myocardial infarction is an early myocardial infarction.

20. The method according to claim 18, wherein the primers are selected from the group consisting of SEQ ID NO: 3 and SEQ ID NO: 4.

21. A method for the in vitro diagnosis of thromboembolic and/or coronary heart diseases comprising the steps of
(a) obtaining a sample from a person, wherein said sanple is selected from the group consisting of a cell, tissue, body fluid, a cellular component of the blood, endothelial cells and smooth muscle cells;
(b) isolating ENGL2 protein from said sample;
(c) determining the amino acid at position 58 of the EGLN2 protein; and
(d) assessing the risk for a thromboembolic and/or coronary heart disease, in particular for an early myocardial infarction, stroke, PRIND, TIA and/or coronary heart diseases wherein an amino acid exchange from serine to lelucin at the position corresponding to position 58 is indicative of increased risk of said disease.

22. (canceled)


SEQUENCE LISTING (4 sequences)

SEQ ID NO: 1 - DNA, 2111 bp, Homo sapiens
gctttcccctgcctgcctgtctctagtttctctcacatccctttttttttttcctttctctagccaccctgaagggtcccttcccaagcccttagggaccgcagaggacttggggaccagcaagcaacccccagggcacgagaagagctcttgctgtctgccctgcctcaccctgccccacgccaggcccggtggcccccagctgcatcaagtggaggcggaggaggaggcggaggagggtggcaccatgggcccgggcggtgccctccatgcccgggggatgaagacactgctgccatggacagcccgtgccagccgcagcccctaagtcaggctctccctcagttaccagggtcttcgtcagagcccttggagcctgagcctggccgggccaggatgggagtggagagttacctgccctgtcccctgctcccctcctaccactgtccaggagtgcctagtgaggcctcggcagggagtgggacccccagagccacagccacctctaccactgccagccctcttcgggacggttttggcgggcaggatggtggtgagctgcggccgctgcagagtgaaggcgctgcagcgctggtcaccaaggggtgccagcgattggcagcccagggcgcacggcctgaggcccccaaacggaaatgggccgaggatggtggggatgccccttcacccagcaaacggccctgggccaggcaagagaaccaggaggcagagcgggagggtggcatgagctgcagctgcagcagtggcagtggtgaggccagtgctgggctgatggaggaggcgctgccctctgcgcccgagcgcctggccctggactatatcgtgccctgcatgcggtactacggcatctgcgtcaaggacagcttcctgggggcagcactgggcggtcgcgtgctggccgaggtggaggccctcaaacggggtgggcgcctgcgagacgggcagctagtgagccagagggcgatcccgccgcgcagcatccgtggggaccagattgcctgggtggaaggccatgaaccaggctgtcgaagcattggtgccctcatggcccatgtggacgccgtcatccgccactgcgcagggcggctgggcagctatgtcatcaacgggcgcaccaaggccatggtggcgtgttacccaggcaacgggctcgggtacgtaaggcacgttgacaatccccacggcgatgggcgctgcatcacctgtatctattacctgaatcagaactgggacgttaaggtgcatggcggcctgctgcagatcttccctgagggccggcccgtggtagccaacatcgagccactctttgaccggttgctcattttctggtctgaccggcggaacccccacgaggtgaagccagcctatgccaccaggtacgccatcactgtctggtattttgatgccaaggagcgggcagcagccaaagacaagtatcagctagcatcaggacagaaaggtgtccaagtacctgtatcacagccgcctacgcccacctagtggccagtcccagagccgcatggcagacagcttaaatgacttcaggagagccctgggcctgtgctggctgctccttccctgccaccgctgctgcttctgactttgcctctgtcctgcctggtgtggagggctctgtctgttgctgaggaccaaggaggagaagagacctttgctgccccatcatgggggctggggttgtcacctggacagggggcagccgtggaggccaccgttaccaactgaagctgggggcctgggtcctaccctgtctggtcatgaccccattaggtatggagagctgggaggaggcattgtcacttcccaccaggatgcaggacttggggttgaggtgagtcatggcctcttgctggcaatggggtgggaggagtacccccaagtcctctcactcctccagcctggaatgtgaagtgactccccaacccctttggccatggcaggcaccttttggactgggctgccactgcttgggcagagtaaaaggtgccaggaggagcatgggtgtggaagtcctgtcagccaagaaataaaagtttacctcagagctgcaaaaaaaaaaaaaaaaaaaaaaa

SEQ ID NO: 2 - protein, 407 aa, Homo sapiens
MDSPCQPQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCSCSSGSGEASAGLMEEALPSAPERLALDYIVPCMRYYGICVKDSFLGAALGGRVLAEVEALKRGGRLRDGQLVSQRAIPPRSIRGDQIAWVEGHEPGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 3 - DNA, 20 bp, Artificial Sequence
  Description of Artificial Sequence primer
ctgtccaggagtgcctagtg

SEQ ID NO: 4 - DNA, 18 bp, Artificial Sequence
  Description of Artificial Sequence primer
gggctggcagtggtagag